(19) United States
(12) Patent Application Publication
(10) Pub. No.: US 2014/0273075 A1
(43) Pub. Date: Sep. 18, 2014

(54) METHODS, SYSTEMS AND DEVICES FOR DETERMINING WHITE BLOOD CELL COUNTS FOR RADIATION EXPOSURE

(71) Applicant: EYE MARKER SYSTEMS, INC., Morgantown, WV (US)

(72) Inventors: Christopher J. Kolanko, Morgantown, WV (US); Wesley O. McGee, McLean, VA (US)

(73) Assignee: EYE MARKER SYSTEMS, INC., Morgantown, WV (US)

(21) Appl. No.: 13/841,841

(22) Filed: Mar. 15, 2013

(51) Int. Cl. G01N 33/50

(52) U.S. Cl. CPC G01N 33/5091
USPC 435/39; 435/288.7; 435/34

(57) ABSTRACT

Systems and methods are provided for imaging at least one eye with optical components and processing the image of the at least one eye to determine radiation exposure. An image of one or more eyes may be received. The systems and methods may distinguish blood cell types in the image; quantify circulating levels of a selected blood cell type; output a number of blood cells per volume of the selected blood cell type; and determine a radiation dose based on the number of blood cells per volume of the selected blood cell type.

### FIELD OF THE INVENTION

The present invention relates to the field of radiation biodosimetry, and more specifically, to methods, systems and devices for determining if an individual has been exposed to radiation and what levels of radiation exposure have occurred based on white blood cell counts.

### BACKGROUND OF INVENTION

Exposure to ionizing radiation can have a negative effect on the human body. Whether this is as a result of accidental or intentional exposure, symptoms can range in severity from the minor, such as fatigue, to the severe, including death. Significant concern over the possibility of a radiological or nuclear attack on the U.S. military personnel and civilian populations is becoming more evident as terror organizations are expanding their global reach and becoming more skilled in accessing the information and materials needed to inflict devastating consequences. The proliferation of nuclear weapons and radiological materials for implementation in a terrorist-related dispersal device is a significant threat not only to military personnel but also to civilian populations. Potential sources of exposure may include the contamination of the food/water supply, the placement of radiation sources in public environments, radiological dispersal device detonation, even attack on a nuclear power plant and its associated “fallout”. The route chosen inevitably impacts the size of the exposed population, but the rapid triaging of those who have been exposed versus those who have not represents a critical step in mitigating the health consequences of such an event. Critical to the preparedness for such an event is the ability to rapidly and accurately assess radiation dose, facilitating triaging and health management. Current methods of radiation biodosimetry employed to triage and determine exposure levels are time consuming (days), frequently invasive, and are mainly conducted by trained personnel within a laboratory. The lack of rapid techniques and technology for field-based dose assessments within hours or days after exposure can negatively impact the success of medical intervention(s) for an exposed individual. There is a critical need for the development of a non-invasive technology that can rapidly and accurately identify those individuals who need treatment from those who do not.

The magnitude of triage after a nuclear incident and the need for rapid radiation dose assessment is illustrated by an event that occurred in Goiania, Brazil in 1987. In Goiania, a teletherapy machine's radiation source was pilfered from an abandoned hospital site, exposing a significant population to radiation as the “interesting item” was passed around and attempted to be opened. As a result, 112,800 people were triaged in the city's soccer stadium from September 30th through December 21st. Through a long and laborious process it was determined that of those triaged individuals 249 people had been exposed with total body doses estimated to be between 4.5 and 6.0 Gy (gray, represents the amount of radiation required to deposit 1 joule of energy in 1 kilogram of any kind of matter) with four people having died from the incident. Among the lessons learned from this accident were that medical triage was long and complex and depended upon symptoms of exposure and classical cellular and cytogenetic assays to determine doses. As evidenced by this event, there exists a critical need for rapid biodosimetry diagnostics assays and technology to address the need of triage in order to aid in clinical management of radiation accident victims.

There are two primary methods currently available for assessing radiation exposure: (1) physical assessment and (2) biological/clinical assessment. Physical assessment primarily relies on whole body radiation dosimetry. This method is sensitive and precise, but requires the utilization of equipment which has limited availability and requires collection of and calibration to additional environmental radiological measurements. In addition to this being a time consuming process, the likelihood of such apparatuses being in place at the site at the time of exposure is highly unlikely. Quantifying the deposition of radiation in various tissues is also a means of physical assessment but, this too is a time intensive process and requires specialized equipment and unlike whole body radiation dosimetry, can be an invasive process.

Since it is unlikely physical dosimeters may be utilized in the scenario of a terrorist event for reasons such as those discussed above, military personnel and first-responders may likely be dependent upon biological/clinical-based evaluation to assess radiation exposure. There are three primary ways to assess radiation exposure by biological and/or clinical means: (1) time to onset of emesis, or vomiting, (2) chromosomal aberrations, and (3) lymphocyte depletion kinetics. When the dosage of radiation exposure exceeds 6 Gray (Gy), the time of vomiting is inversely proportional to the absorbed radiation dosage. At exposures less than 4 Gy, however, vomiting is less common and cannot be used as a reliable estimate of radiation dose. The frequency of (lymphocyte) chromosomal aberrations, such as the formation of dicentrics (the interchange between two chromosomes) and chromosomal ring formations (the breakage of an arm of a single chromatid followed by its rejoining to form a ring and a fragment), can correlate well with radiation dose but can easily require 4-5 days of processing to obtain results and are expensive to complete. Additionally, the monitoring of silent or dysfunctional genes, such as the hypoxanthine-guanine phosphoribosyltransferase gene (hprt), is frequently used to monitor (radiation-induced) genetic mutations but are, unfortunately, limited by the high degree of variance found amongst the population as well as the extensive processing time required to obtain test results, sometimes upwards of two weeks. It has been shown that lymphocyte depletion kinetics are radiation dose-related (FIG. 1) and can be used to estimate radiation exposure between 1 and 10 Gy photon equivalent dose range. FIG. 1 shows a classical Andrew's lymphocyte depletion curves with accompanying clinical severity ranges. Curves 1-4 correspond roughly to the following whole-body doses: 3.1 Gy (curve 1); 4.4 Gy (curve 2); 5.6 Gy (curve 3); 7.1 Gy (curve 4). Normal lymphocyte numbers in healthy individuals range between 15,000 cells/mL and 35,000 cells/mL and a radiation dose as low as 0.2 Gy can cause lymphocyte death resulting in an observable depletion in the absolute count. Additionally, an algorithm has been developed to estimate whole-body doses in the range of 0.5-10 Gy based upon the rate of decline of circulating lymphocytes. Ideally, monitoring all three biological parameters (time to onset of emesis, chromosomal aberrations, and lymphocyte depletion kinetics) provides the most complete clinical picture of radiation exposure. Practically speaking, however, only the time to the onset of vomiting and lymphocyte depletion kinetics is amenable to monitoring within the first 24 hours after exposure. As previously mentioned, a narrower range of utility exists when monitoring the time to onset of vomiting when compared to the monitoring of lymphocyte depletion kinetics.

Only if laboratory capacity remains intact and is not overwhelmed by a mass casualty exposure, tracking the rate and magnitude of decline in absolute lymphocyte counts over a period of hours to days serves as the single best estimator of radiation exposure and clinical outcome. Depending on the absorbed dose, such changes can begin within hours of exposure, so current recommendations are to perform a complete blood cell count with differential as a baseline right away, and then every 6-12 hours thereafter for 2-3 days. FIG. 2 shows a time-dependent response model of the various peripheral blood cell components to a 1 Gy and 3 Gy whole body exposures. As illustrated in FIG. 2, lymphocytes respond the earliest to radiation exposure when compared to the other blood cell types.

The advantages of using lymphocyte depletion kinetics as an early indicator of radiation exposure during a mass casualty event are listed below.

Lymphocyte depletion kinetics
Can be used for both whole- or partial-body acute radiation exposure over minutes to hours
Serial blood cell counts are obtained and the absolute lymphocyte count is calculated and tracked over time

Lymphocyte depletion rate is directly related to radiation absorbed dose
2-4 Gy: lymphocyte decline occurs over ˜4-6 days
4-6 Gy: lymphocyte decline requires ˜2-4 days
At sufficiently high radiation doses, lymphocyte decline may be measurable from day 1 post-exposure.

Lymphocyte depletion kinetics can be used
To provide an early/preliminary estimate of radiation dose
To guide initial clinical management and triage of the patient, radiation dose estimates based on lymphocyte depletion kinetics should be used in conjunction with all other clinical and laboratory information
In large scale radiation events, biodosimetry based on lymphocyte depletion kinetics, clinical signs and symptoms, and dose reconstruction from geographic information are likely to be available more rapidly than biodosimetry based on dicentric chromosome assay.

Traditionally, the monitoring of lymphocyte depletion kinetics is performed by the acquisition and subsequent analysis of a peripheral blood sample. While there are a myriad of platforms which can be used to analyze such a sample, there are several things that are typically shared amongst these. In addition to the significant footprint of such equipment, its sensitivity to movement and its delicate calibrations, the costs associated with its acquisition, and the specialized training required to operate and maintain it, analysis requires the semi-invasive acquisition of a blood sample, which is associated with its own risks even when performed by trained/certified individuals in the best of settings. The hazards associated with blood sample acquisition, in addition to those posed to the donor, include the possible spread of infectious disease and stringent sample disposal regulations. Common elements such as these found among current state of the art technology makes their routine portability and field applicability questionable. The ability to perform blood cell analysis, such as lymphocyte depletion kinetics, in a variety of environments without subjecting persons to possible blood-related hazards, in a rapid, accurate, cost effective, and reproducible manner is of utmost importance in advancing our ability to respond to terror threats such as unleashed radiation exposure.

The Biological Assessment Tool, available through the Armed Forces Radiobiology Research Institute (AFRRI), is a computational tool currently used to estimate individual radiation dose based upon clinical signs and symptoms, such as those outlined by the International Atomic Energy Agency and in the Medical Treatment Protocols. Specialized cytogenetic biodosimetry laboratories and mobile hematology laboratories process peripheral blood samples to determine chromosomal aberrations and lymphocyte depletion kinetics, respectively, in the case of a mass-casualty scenario.

As part of the National Strategic Plan and Research Agenda for Medical Countermeasures against Radiological and Nuclear Threats, the development of new countermeasure products that may be used in a public health emergency are of critical importance. The ability to perform radiation dose assessment, such as lymphocyte depletion kinetics, in a myriad of environments without subjecting persons to possible blood-related hazards, in a rapid, accurate, cost effective, and reproducible manner is of utmost importance in advancing our ability to respond to terror threats such as unleashed radiation exposure. This diagnostic assay fills an important need for the Strategic National Stockpile (SNS).

The proliferation of nuclear weapons and radiological materials for implementation in a terrorist-related device is a significant threat to both military personnel and the US population. A nuclear detonation and/or release of radioactive material by a terrorist group could result in a number of individuals being exposed to ionizing radiation. There would also be a significant amount of contamination within urban centers, leading to economical and social disruption.

No technologies currently exist that can be used to rapidly triage a large group of individuals potentially exposed to ionizing radiation. The current methods of radiation biodosimetry employed to triage and determine exposure levels are time consuming (days) and are mainly conducted by trained personnel within a laboratory. Current cytogenetic methods such as dicentric chromosome assays take upwards of 72 hours to perform before exposure levels are determined.

New methods for determining radiation exposure are being developed that measure protein biomarkers in blood or the level of oncogene expression. Though these techniques are faster to perform in comparison to cytogenetic-based assays, they are still invasive and require a high level of scientific expertise and instrumentation that is not feasible for field-deployable triage. This non-availability of rapid techniques and technology for dose assessments within hours or days after exposure could result in the sub-optimal medical treatment for an exposed individual. The desired diagnostic technology must be relatively simple to use (requiring minimal training), is preferably non-invasive, and can be utilized at the point-of-care.

To reliably and non-invasively detect exposure to ionizing radiation, it is important to employ a target organ, tissue, or source that may signal deleterious effects to a physiological system. One such accessible source is the eye. Over the past century, epidemiological findings and several scientific studies have identified the eye as a prime indicator for a vast array of diseases, syndromes, abnormalities and exposures. Numerous physiological insults lead to identifiable, differential markers in the eyes due to their unique interaction with the multiple physiological systems to which the eyes are interconnected. Consequently, once properly defined, characterized, and quantified, such ocular characteristics may be employable for diagnostic purposes, such as connecting the manifestation of an exposure to a particular dose.

Needs exist for new methods, systems and devices for reliably and non-invasively detecting exposure to ionizing radiation.

### SUMMARY OF INVENTION

Embodiments of the present invention may provide a rapid and accurate assessment of radiation dose by monitoring white blood cells within hours of potential exposure, thereby improving triaging and medical management of identified victims and reducing associated morbidity and mortality.

Certain embodiments of the present invention may include a system for determining radiation exposure. The system may include at least one processor and memory. The at least one processor may receive an image of one or more eyes; distinguish blood cell types in the image; quantify circulating levels of a selected blood cell type; output a number of blood cells per volume of the selected blood cell type; and determine a radiation dose based on the number of blood cells per volume of the selected blood cell type. The system may also have optical components for imaging the one or more eyes, and may be handheld. The blood cell types may be lymphocytes. The processor may calculate blood volume within a vessel shown in the image. The distinguishing may include differentiating between red and white blood cells. The quantifying may include identifying and calculating white blood cell types. The processor may calculate the number of cells of a specified blood cell type per volume of blood and compare the number of blood cells per volume to a standard to determine the radiation dose.

In embodiments of the present invention, a system for determining radiation exposure may include optical components for imaging an eye, and at least one processor and memory. The processor may obtain data regarding one or more eyes with the optical components; determine ocular blood vessels in the data from the optical components; differentiate white blood cells within the ocular blood vessels; identify peripheral blood lymphocytes; quantify lymphocytes within a specified volume of circulating blood; and correlate the resulting quantification to a specified dose of radiation. The system may be handheld. The optical components may be a spectroscopic probe or optical imagers. The correlating may be completed with five minutes of receiving the image.

Certain embodiments of the present invention may include a method of determining radiation exposure. The method may include imaging at least one eye with optical components; determining one or more blood vessels in the image; differentiation blood cell types in the image; determining a quantity of a specified blood cell type per volume of blood; and correlating the quantity of the specified blood cell type per volume of blood to a specific radiation dose. The optical components and processor may be in a portable, handheld device. The imaging may include holding a portable, handheld device to a user's one or more eyes for imaging. The user may be directed to look at a focal point for a specified period of time. The processor may determine a ratio of different blood cell types. The imaging may be spectral imaging.

Additional features, advantages, and embodiments of the invention are set forth or apparent from consideration of the following detailed description, drawings and claims. Moreover, it is to be understood that both the foregoing summary of the invention and the following detailed description are exemplary and intended to provide further explanation without limiting the scope of the invention as claimed.

### BRIEF DESCRIPTIONS OF THE FIGURES

The accompanying drawings, which are included to provide a further understanding of the invention and are incorporated in and constitute a part of this specification, illustrate preferred embodiments of the invention and together with the detailed description serve to explain the principles of the invention. In the drawings:

FIG. 1 shows a classical Andrew's lymphocyte depletion curves with accompanying clinical severity ranges.

FIG. 2 shows a time-dependent response model of the various peripheral blood cell components to a 1 Gy and 3 Gy whole body exposures.

FIG. 3 is a schematic of ocular medical radiation non-invasive diagnostic.

FIG. 4 shows exemplary embodiments of systems for the diagnosis of biological conditions.

FIG. 5 shows phenotypic characteristics of peripheral blood cells.

### DETAILED DESCRIPTION OF THE EMBODIMENTS

Embodiments of the present invention may take advantage of (1) divergent blood cell morphologies between red and white blood cells and external ocular blood vessels, (2) the readily visible superficial ocular blood vessels lying above the sclera (white portion) of the eye, and/or (3) the well-characterized internal retinal blood vessels for development of a diagnostic capability for radiation exposure. Cell morphologies may include the size, shape and internal features that differentiate between red and white blood cells and different types of white blood cells.

Ocular blood vessels exhibit a number of favorable characteristics that may increase the feasibility of microscopic blood cell analysis. For example, these vessels are close to the outside of the body (minimizes observation working distance), are readily visible/imageable, numerous, of ample size (up to about 100 μm) for imaging, and the background provides a high contrast for vessel visualization.

Subsequent to spectroscopic probe or image acquisition, algorithms can be employed to distinguish and quantify the circulating levels of blood cell types such as lymphocytes. Based on captured data and/or images from one or more blood vessels, the algorithms may (1) calculate a blood volume within the one or more blood vessels, (2) differentiate between cell types, such as red and white blood cells, (3) identify and quantify a certain blood cell type, such as white blood cells generally and/or specific types of white blood cells, such as lymphocytes, and (4) calculate a number of cells per volume of blood. Device output may be the number of cells per blood volume.

Embodiments of the present invention may include a non-invasive diagnostic assay system for exposure to ionizing radiation (1) using one or more blood vessels, preferably ocular blood vessels, to spectroscopically probe or optically image and differentiate blood cell types, such as white blood cells, (2) using an imaging system to obtain requisite data and/or images rapidly enough and with sufficient resolution to identify selected cell types, such as peripheral blood lymphocytes, and (3) using an algorithm that permits quantification of the selected cell types, such as lymphocytes within a specified volume of circulating blood and correlates this number to a specified dose of radiation. Preferably, this analysis is completed within a few minutes of obtaining image or other data, preferably less than ten minutes, more preferably less than five minutes, more preferably less than three minutes, and more preferably less than one minute.

The diagnostic assay system of the present invention is an advancement in non-invasive blood cell counting that may allow for a complete point-of-care medical radiation diagnostic system through the application of an integrated optical technology and detection/quantification algorithms. Through direct visualization of ocular blood vessels via advanced imaging technology, a non-invasive lymphocyte depletion diagnostic assay system may screen individuals for absorbed ionizing radiation exposure. This system may have the capability to triage a large population of individuals for radiation exposure. The medical radiation diagnostic capability can be integrated into the Department of Defense “System-of-systems” approach by providing diagnostic capabilities to triage the warfighter and civilian populations exposed to ionizing radiation. As medical countermeasures administered for radiation exposure are not easily assessable, and in some case can be toxic, there is critical need to provide diagnostic capabilities that can triage those individuals that need immediate attention.

The diagnostic assay system of the present invention may measure radiation exposure dose. The device may be held to the patient's eye for imaging. Alternatively, the device may be a stationary device that the user places their eye in proximity to or in contact with. The image may be acquired and analyzed to determine a ratio of specific blood cells. Analysis may be done by software that measures blood volume and cell counts, such as white blood cell counts. The ratio of blood cells to blood volume may then be determined and correlated to a specific radiation dose.

Multiple approaches may be used to determine a ratio of cell types, such as a ratio of white blood to red blood cell. In certain embodiments, (1) spectroscopic techniques, and (2) direct imaging may be used. Spectroscopic techniques may be performed by specific imaging cameras. Similar to an in vitro blood cell counter, an ocular laboratory imaging technology for direct imaging may microscopically visualize the eye blood vessels and specific cells in circulation. Laboratory optical systems may operate at a magnification necessary to spectroscopically probe individual vessel flows or optically image individual cells within a given vessel.

Spectroscopic signatures of red and white blood cells in vivo may be evaluated by embodiments of the present invention. These may include the absorption of the different cell types by different wavelengths of light. Hardware associated with obtaining these signatures may include fluorescence cameras. Although optical means of blood cell quantitation employing light scattering methods on stationary vials or measuring the fluorescence of labeled cells in vivo have shown good results, neither of these methodologies is an acceptable method for this study of unlabeled cells flowing at normal capillary flow velocities (0.1-1.4 mm/s). In embodiments of the present invention, methods may exploit optical interactions of blood cells with different sources of illumination at optimum absorption wavelengths such as hyper-spectral imaging. Hyper-spectral uses a prism to spatially separate spectral components of the incoming image and capture all of them simultaneously. This system may work well for moving images, allowing co-registration and tracking of objects in the field of view. In addition, this hyper-spectral imaging method may simplify white blood cell imaging and counting. Specific features within the image can then be differentiated due to varying absorption/reflection wavelengths of items in the image.

Hyper-spectral imaging optical components can be integrated into a portable, handheld system. This system may image one or more eyes and may run analysis algorithms to detect blood vessels, differentiate blood cell types and count specified cells lymphocytes within a volume of blood. Eyes may be images and/or analyzed in parallel or in series. The imaging device may contain multiple components including the lenses, filters, condensers, illumination configuration as well as the camera. The individual components may be commercially available.

Embodiments of the present invention may also include algorithms to distinguish and identify white blood types, such as lymphocytes, found in ocular images. Using computer algorithms (image processing, pattern recognition and statistical analysis software) to automate the detection, characterization, identification (categorization) and statistical analysis of blood cell types may lead to faster and more consistent characterization of the blood cell population than human visual inspection and manual recording—with less chance for user bias and less reliance on user expertise. In existing systems, human blood cell counting involves the visual inspection and counting of specific cells on microscope slides. This process is done by trained individuals under laboratory conditions. In embodiments of the present invention, the process may be automated and run by one or more processors on a computer system and/or server. The automated process may use image recognition software or similar systems to determine the number of blood cells of the specified type.

Algorithms may be based upon cell discrimination. This process may involve identifying an imagery object as a cell and then attempting to identify the cell type. There are multiple features of (unstained) cells that distinguish a lymphocyte cell from myeloid and red blood cells, including cell size, nuclear shape, and cytoplasmic volume. Lymphocytes are (1) approximately round with a (2) large, round central nucleus and (3) a large nucleus:cytoplasm ratio. Myeloid cells are (1) usually round or slightly irregular with a (2) frequently lobed nucleus and (3) a small nucleus:cytoplasm ratio. Red blood cells are (1) small in size compared to white blood cells and (2) do not have a nucleus. Under hyper-spectral imaging cell types can also be differentiated based on their absorption of different wavelengths of light.

The image processing algorithms may be used to perform the following operations: image enhancement, object detection/segmentation, object recognition and results output. In this regard, various digital signal-processing methods may be surveyed to determine the most suitable method for image analysis. The noise in the image may be mitigated by designing adaptive filters whose parameters are learnt based on implicit image characteristics. Initial processing may involve dark-field subtraction and flat-field renormalization to account for CCD (charge couple device) variations, non-uniform lighting and other persistent inter-pixel variations. Subsequent processing may involve global filters, such as histogram equalization to enhance contrast and median or low-pass filters to reduce high-frequency noise (but not true cytoplasmic granularity). A gradient (Laplacian of Gaussian) filter may be applied to enhance edges and other line features used to guide segmentation and demarking regions of interest (like vessel walls).

The process of detecting cells, nuclei and large granules may involve a hierarchy of segmentation schemes based on color and/or relative luminance (greyscale) threshold values. Dilation and erosion techniques may be used remove or soften boundary artifacts (e.g., cell membranes), fill voids, remove speckles, and help isolate segments. Hough transforms may be needed to help detect and isolate specific shapes (circles, ovals, etc.) to further guide segmentation, especially where overlaps (occlusion) and unions (with non-detectable boundaries) are present.

After segmentation, perimeters (convex hulls) and their areas may be computed to get the absolute and relative sizes of cells, nuclei and granules. Area-to-perimeter ratios may be used to gauge their roundness. Gradient filters may be applied to segment interiors and used to gauge texture roughness and relative granularity. Greyscale histograms (of 5-10 bins) of cell interiors may be used to generate descriptor vectors and used along with the other morphological parameters described above to develop a classification system.

A diagnostic assay method according to an embodiment of the present invention may include having a (1) subject place their face to the device, (2) look at a focal point for a specified period of time, (3) remove face from device as directed by the operator or device, and (5) wait for device output.

FIG. 3 is a schematic of ocular medical radiation non-invasive diagnostic.

FIG. 4 shows exemplary embodiments of systems for the diagnosis of biological conditions. A user may place one or more eyes within a dark adjusted housing with a facial interface. An operator may hold the device and operate a switch. Imaging components may be housed in the device. A display may provide results and/or data may be sent to another device for processing and/or display.

FIG. 5 shows phenotypic characteristics of peripheral blood cells.

Although the foregoing description is directed to the preferred embodiments of the invention, it is noted that other variations and modifications will be apparent to those skilled in the art, and may be made departing from the spirit or scope of the invention. Moreover, features described in connection with one embodiment of the invention may be used in conjunction with other embodiments, even if not explicitly stated above. The present invention may be embodied in other specific forms without departing from its spirit or essential characteristics. The described embodiments are to be considered in all respects only as illustrative and not restrictive.

1. A system for determining radiation exposure, the system comprising:
at least one processor and memory;
wherein the at least one processor:
receives an image of one or more eyes;
distinguishes blood cell types in the image;
quantifies circulating levels of a selected blood cell type;
outputs a number of blood cells per volume of the selected blood cell type; and
determines a radiation dose based on the number of blood cells per volume of the selected blood cell type.

2. The system of claim 1, further comprising optical components for imaging the one or more eyes.

3. The system of claim 2, wherein the system is handheld.

4. The system of claim 1, wherein the blood cell types are lymphocytes.

5. The system of claim 1, further comprising calculating blood volume within a vessel shown in the image.

6. The system of claim 1, wherein the distinguishing comprises differentiating between red and white blood cells.

7. The system of claim 6, wherein the quantifying circulating levels of blood cell types comprises identifying and calculating white blood cell types.

8. The system of claim 1, further comprising calculating the number of cells of a specified blood cell type per volume of blood.

9. The system of claim 1, further comprising comparing the number of blood cells per volume to a standard to determine the radiation dose.

10. A system for determining radiation exposure, the system comprising:
optical components for imaging an eye;
at least one processor and memory;
wherein the at least one processor:
obtains data regarding one or more eyes with the optical components;
determines ocular blood vessels in the data from the optical components;
differentiates white blood cells within the ocular blood vessels;
identifies peripheral blood lymphocytes;
quantifies lymphocytes within a specified volume of circulating blood; and
correlates the resulting quantification to a specified dose of radiation.

11. The system of claim 10, wherein the system is handheld.

12. The system of claim 10, wherein the optical components are a spectroscopic probe.

13. The system of claim 10, wherein the optical components are optical imagers.

14. The system of claim 10, wherein the correlating is completed with five minutes of receiving

15. A method of determining radiation exposure, the method comprising:
imaging at least one eye with optical components;
determining one or more blood vessels in the image;
differentiation blood cell types in the image;
determining a quantity of a specified blood cell type per volume of blood; and
correlating the quantity of the specified blood cell type per volume of blood to a specific radiation dose.

16. The method of claim 15, wherein the optical components and processor are in a portable, handheld device.

17. The method of claim 15, wherein the imaging comprises holding a portable, handheld device to a user's one or more eyes for imaging.

18. The method of claim 17, wherein the user is directed to look at a focal point for a specified period of time.

19. The method of claim 15, further comprising determining a ratio of different blood cell types.

20. The method of claim 15, wherein the imaging is spectral imaging.
